(19) United States
(12) Patent Application Publication
(10) Pub. No.: US 2013/0122090 A1
(43) Pub. Date: May 16, 2013

(54) Multiple Unit Tablet Composition

(71) Applicants: Sunil Shantwan Borude, Pune (IN); Shrenik Annasaheb Kole, Pune (IN); Makrand Krishnakumar Avachat, Pune (IN)

(72) Inventors: Sunil Shantwan Borude, Pune (IN); Shrenik Annasaheb Kole, Pune (IN); Makrand Krishnakumar Avachat, Pune (IN)

(73) Assignee: LUPIN LIMITED, Mumbai (IN)

(21) Appl. No.: 13/811,327

(22) PCT Filed: Jul. 18, 2011
(86) PCT No.: PCT/IB2011/001658

§ 371 Date: Jan. 21, 2013

(30) Foreign Application Priority Data
Jul. 22, 2010 (IN) 806/KOL/2010

(51) Int. Cl. A61K 9/28; A61K 31/4439; A61K 9/20

(52) U.S. Cl. CPC A61K 9/28; A61K 9/2095; A61K 31/4439
USPC 424/465; 514/341

(57) ABSTRACT

A multiple unit tablet composition comprising an enteric coated multiple unit cores comprising a pharmaceutically active ingredient, wherein plasticizer content of enteric coating is less than about 10% by weight of the enteric coating polymer; at least two diluents and optionally one or more other pharmaceutically acceptable excipient, wherein one diluent is highly compactable microcrystalline cellulose and process for preparing the same.

### FIELD OF THE INVENTION

The present invention relates to multiple unit tablet composition comprising a pharmaceutically active ingredient and a process for preparing them.

### BACKGROUND AND PRIOR ART OF THE INVENTION

Multiple unit dosage forms are multiparticulate drug delivery systems consisting of plurality of pellets, granules, spherules, micro spheres, mini tablets and other drug substance containing agglomerations or particles that can be loaded into either a capsule or tablet. Single unit dosage forms are traditional tablets and powder-filled capsules.

Multiple unit dosage forms offer numerous significant therapeutic advantages over traditional single unit dosage forms, that these generally disperse freely in the gastrointestinal tract and behave like liquids, leaving the stomach within a short period of time, which results in:
improved biopharmaceutical characteristics, such as improved bioavailability, reduced the effect of the food on plasma profiles and in such way reduced intra- and inter-subject variability of plasma profiles,
reduced peak plasma fluctuations and in such way minimized potential side effects,
minimized local irritation in gastrointestinal tract.

When formulated as modified release dosage forms, multiple units perform improved safety and efficacy, since they are less susceptible to dose dumping than single unit formulations with modified release. Multiple unit dosage forms are commonly filled into hard capsules. The alternative method of formulating multiple units is compacting into multiple unit tablets. This approach becomes increasingly important in pharmaceutical industry because of combining the advantages of multiple unit dosage forms and tablets in one final dosage form.

Two main challenges of formulating of multiple units into multiple unit tablets are:
Tableting of multiple units into multiple unit tablets is mechanically stressful process for multiple units that are likely to be mechanically damaged during compression step of tableting. The damage of the multiple units during compression step of tableting can result in altered drug release. This is especially critical when multiple units coated with functional coating that assures modified release are tableted into multiple unit tablets, because compression step of tableting often results in rupture of functional coating.

During the compression step of tableting of pellets with tableting excipients the particle segregation tends to occur, because of different size and shape of multiple units and particles of excipients. The particle segregation results in inappropriate content and mass uniformity of multiple unit tablets.

Therefore, two main requirements that must be met when formulating multiple unit tablets are that drug release from multiple unit tablets is not altered compared to the drug release from multiple units prior to tableting, and that the content and mass uniformity of multiple unit tablets complies with pharmacopoeia prescriptions.

Additionally, multiple unit tablets must be coherent and have appropriate hardness and friability, so that they could be further handled, such as coated and packed.

There are two approaches in multiple units tableting:
tableting of multiple units without other excipients for tableting, and
tableting of multiple units together with pharmaceutically acceptable excipients.

The approach of compacting of only multiple units without other excipients for tableting does not include the problem of particle segregation, but includes very difficult formulation of multiple units and the coating of multiple units. Multiple unit cores must be deformable enough so that they form coherent tablets, and the coatings of multiple units must be able to withstand compacting without damages, which can be ensured by formulating the coating of multiple units in such way that the coating posses improved elasticity.

The approach of compacting of multiple units together with pharmaceutically acceptable tableting excipients moderates requirements for the multiple units coating elastic properties, since plastically deformable or soft tableting excipients are able to partly absorb compaction forces and protect multiple units from mechanical damages. This approach enables also easier obtaining of coherent multiple unit tablets that have appropriate hardness and friability, because tableting excipients have larger surface area than subunits and better bonding inside multiple unit tablets.

However, tableting of multiple units together with pharmaceutically acceptable tableting excipients includes problem of particle segregation. Particle segregation in the tableting mixture results in tableting problems, such as weight variation and poor content uniformity.

U.S. Pat. No. 5,753,265 relates to an oral pharmaceutical composition in the form of a multiple unit tablet comprising: a tablet excipient; a multiple of a core unit comprising as an active ingredient an acid-labile H⁺K⁺-ATPase inhibitor compound in a neutral form or a salt form, a single enantiomer or an alkaline salt of a single enantiomer; the core unit being covered with at least one enteric coating layer having mechanical properties so as not to significantly affect the acid resistance of the enteric coating layered unit by compression during tableting.

WO 2008/047320 relates to multiple unit tablet compositions of benzimidazole compounds and process of preparation thereof. The compositions are useful against various gastrointestinal disorders. The multiple unit tablet composition comprises: a) tablet excipients, and b) multiple enteric coated core units containing a benzimidazole compound, wherein each core unit is covered with an enteric coating layer comprising a plasticizer in an amount of less than 15% by weight of the enteric coating layer polymer.

WO 2008/006534 discloses multiple unit tablets comprising multiple units compacted together with at least two tablet filler-binders and optionally other pharmaceutically acceptable excipients, wherein at least one of said tablet filler-binder is a tablet filler-binder having mean particle size-to-mean multiple unit size ratio from 10% to 40%, and at least one of said tablet filler-binder is a tablet filler-binder having mean particle size-to-mean multiple unit size ratio from 1% to 10%.

It has been surprisingly found by the inventors that robust multiple unit tablet compositions having excellent mechanical properties of enteric coated multiple unit cores without damaging or rupturing during the compression, further without altering the acid resistance can be prepared by using atleast two diluents, wherein one diluent is highly compactible microcrystalline cellulose along with enteric coated multiple unit cores, wherein the plasticizer content of enteric coating layer is less than about 10% by weight of enteric coating polymer.

### OBJECTS OF THE INVENTION

According to one embodiment a multiple unit tablet composition comprising:

an enteric coated multiple unit cores comprising a pharmaceutically active ingredient, wherein plasticizer content of enteric coating is less than about 10% by weight of the enteric coating polymer,

at least two diluents and optionally one or more pharmaceutically acceptable excipient(s), wherein one diluent is highly compactible microcrystalline cellulose.

According to another embodiment a multiple unit tablet composition comprising:

(i) a enteric coated multiple unit cores comprising a pharmaceutically active ingredient, wherein plasticizer content of enteric coating is less than about 10% by weight of the enteric coating polymer,

(ii) atleast two diluents and optionally one or more pharmaceutically acceptable excipient(s), wherein one diluent is highly compactible microcrystalline cellulose, wherein the multiple units of the active ingredient are about 20-45% of the tablet weight.

According to yet another embodiment a multiple unit tablet composition comprising:

(i) enteric coated multiple unit cores comprising a pharmaceutically active ingredient, wherein plasticizer content of enteric coating is less than about 10% by weight of the enteric coating polymer,

(ii) atleast two diluents and optionally one or more pharmaceutically acceptable excipient(s), wherein one diluent is highly compactible microcrystalline cellulose, wherein the multiple units of the active ingredient are about 20-45% of the tablet weight, and the multiple unit tablet composition has mechanical properties wherein the acid resistance is not compromised.

According to yet another embodiment a multiple unit tablet composition comprising:

(i) enteric coated multiple unit cores comprising a pharmaceutically active ingredient, wherein plasticizer content of enteric coating is less than about 10% by weight of the enteric coating polymer,

(ii) atleast two diluents, one or more stabilizers and optionally one or more pharmaceutically acceptable excipient(s), wherein one diluent is highly compactible microcrystalline cellulose, wherein the multiple units of the active ingredient are about 20-45% of the tablet weight, and the multiple unit tablet composition has mechanical properties wherein the acid resistance is not compromised.

According to yet another embodiment a process for the preparation of multiple unit tablet composition comprising the steps of mixing enteric coated multiple unit cores of active ingredient having plasticizer content of less than about 10% by weight of the enteric coating polymer with atleast two diluents having highly compactible microcrystalline cellulose as one diluent and one or more other pharmaceutically acceptable excipients and compressed.

### SUMMARY OF THE INVENTION

One aspect of the present invention embodies a multiple unit tablet composition comprising:
(i) an enteric coated multiple unit cores comprising a pharmaceutically active ingredient, wherein plasticizer content of enteric coating is less than about 10% by weight of the enteric coating polymer;
(ii) atleast two diluents and optionally one or more other pharmaceutically acceptable excipient, wherein one diluent is highly compactable microcrystalline cellulose.

Yet another aspect of the invention embodies a process for the preparation of multiple unit tablet composition comprising the steps of mixing enteric coated multiple unit cores of active ingredient having plasticizer content of less than about 10% by weight of the enteric coating polymer with, atleast two diluents having high compactible microcrystalline cellulose as one diluent and one or more other pharmaceutically acceptable excipients and compressed.

### DETAILED DESCRIPTION OF THE INVENTION

According to one embodiment a multiple unit tablet composition comprising:

an enteric coated multiple unit cores comprising a pharmaceutically active ingredient, wherein plasticizer content of enteric coating is less than about 10% by weight of the enteric coating polymer, atleast two diluents and optionally one or more pharmaceutically acceptable excipient(s), wherein one diluent is highly compactible microcrystalline cellulose

The pharmaceutically active ingredient comprised in multiple units in multiple unit tablets according to the present invention may be selected from the group consisting of analgesics, anticonvulsants, antiparkinsonics, anesthetics, antibiotics, antihypertensives, antihistaminics, antimalarial agents, antimigraine agents, anti-obesity agents, serum lipid reducing agents, antipyretics, alpha-blockers, alpha-adrenergic agonists, bactericides, bronchial dilators, beta-adrenergic stimulants, beta-adrenergic blockers, enzymes, contraceptives, cardiovascular active substances, calcium channel inhibitors, proton pump inhibitors, diuretics, hypnotics, hormones, hyperglycemics, hypoglycemics, muscle relaxants and contractors, parasympathomimetics, sedatives, sympathomimetics, tranquillizers, vitamins or their pharmaceutically acceptable salt, polymorph, solvate(s), hydrate(s), enantiomer(s) and any combinations thereof. Preferably, the pharmaceutically active ingredient is proton pump inhibitor, such as omeprazole, esomeprazole, lansoprazole, rabeprazole, pantoprazole or their pharmaceutically acceptable salt, polymorph, solvate(s), hydrate(s), enantiomer(s) thereof.

One or more pharmaceutically acceptable excipient(s) are but not limited to binders, diluents, disintegrants, surfactants (solubilizers/wetting), lubricants/glidants. One excipient can perform more than one function.

The diluents may be selected from hydrogenated vegetable oil, one or more of sugars like dextrose, glucose, lactose; sugar alcohols like sorbitol, xylitol, mannitol; cellulose derivatives like powdered cellulose, microcrystalline cellulose; starches like corn starch, pregelatinized starch, maize starch and mixtures thereof. Two grades of the same diluent can also be used.

The one of the diluent used in the present invention acts as a means of direct compression by providing high compactibility, the most preferable diluent used is but not limited to Microcrystalline cellulose i.e CEOLUS™ KG. CEOLUS™ KG has extraordinary compactibility and is a maximum-compactibility MCC powder with Rodform particles.

The unparalleled compactibility of CEOLUS™ KG originates in its distinctive rodform particles. This compactibility enables, in particular:
Reduced MCC addition and tablet size.
Reduced compression force and superior tableting of pressure-sensitive enzymes, antibiotics, and film-coated granules.

We have surprisingly found that by using CEOLUS™ KG as one of the diluents helps in reducing the plasticizer content in the enteric layer to about less than 10% further without compromising the mechanical properties of enteric coated multiple unit cores i.e without damaging or rupturing during the compression, thereby not altering acid resistance.

In addition to mechanical properties, CEOLUS™ KG also helps in making robust tablets having appropriate hardness and friability so that they could be further coated and packed, it has being found that the friability of the composition of the composition is less than 0.15% or most preferably nil.

The binders are selected from but not limited to one or more of cellulose derivatives like hydroxypropylmethyl cellulose, hydroxypropyl cellulose, methylcellulose; gums like xanthan gum, gum acacia, tragacanth; water-soluble vinylpyrrolidone polymers like polyvinylpyrrolidone, copolymer of vinylpyrrolidone and vinyl acetate; sugars like sorbitol, mannitol and mixtures thereof.

The disintegrants are selected from but not limited to one or more of croscarmellose sodium, carmellose sodium, carmellose calcium, crospovidone, sodium starch glycolate, low-substituted hydroxypropyl cellulose, hydroxypropyl starch, crospovidone, cornstarch and mixtures thereof.

The lubricants/glidants are selected from but not limited to one or more of magnesium stearate, stearic acid, sodium stearyl fumarate, calcium stearate, zinc stearate,colloidal silicon dioxide or mixture thereof.

The surfactant may be selected from but not limited to one or more of sodium lauryl sulphate, polysorbate 80, Polaxmer, DSS (dioctyl sodium sulfosuccinate), triethanolamine, sodium lauryl sulphate, polyoxyethylene sorbitan and poloxalkol derivatives, quaternary ammonium salts or mixtures thereof or other pharmaceutically acceptable surface-active agents known to one ordinary skilled in the art. The surfactant further helps in improving the solubility and bioavailability of the composition.

The core may be in the form of pellets, granules, beads or inert core. The core may be acidic, alkaline or neutral depending on the type of Composition. The core may contain one or more pharmaceutically acceptable excipients selected from the group consisting of inert carriers, binders, diluents, disintegrants, lubricants/glidants, solubilizers/wetting agents and mixtures thereof. In the inert core the inert carrier may be coated with the proton pump inhibitor and one or more of the binders, diluents, disintegrants, lubricants/glidants, solubilizers/wetting agents and mixtures thereof.

The inert carrier may comprise starch, microcrystalline cellulose or sugar sphere such as nonpareil sugar seeds. The inert carrier may be further hardened by preparing the binder solution in water or alcohol or hydroalcoholic solvents and spraying the binder solution on the inert carrier. The hardening solution has a binder, active ingredient and optionally one or more pharmaceutically acceptable excipient. The hardened inert carrier further helps in providing robustness to the composition.

The core may be coated with a separating layer prior to the enteric coating layer. The separating layer is made up of water-soluble material, which is capable of dissolving or forming a gel in contact with water. Such material may include water-soluble polymer and/or water-soluble substance. The water-soluble substance may be selected from but not limited to glucose, lactose, mannitol, sorbitol, sucrose, dextrose and mixtures thereof. The water-soluble polymers may be selected from but not limited to hydroxypropylmethylcellulose, hydroxypropyl cellulose, polyvinylpyrrolidone, sodium alginate, sodium carboxymethyl cellulose, copolymer of vinylpyrrolidone and vinyl acetate.

An enteric coating layer is applied onto the core coated with the separating layer. The enteric coating layer may include polymers such as but not limited to cellulose acetate phthalate, hydroxypropylmethyl cellulose phthalate, polyvinyl acetate phthalate, carboxymethylethylcellulose, methacrylic acid copolymers, for example, compounds known under the trademarks of Eudragit NE30D, Eudragit L, Eudragit S, Eudragit L 100 55 or any combinations and mixtures thereof.

The enteric coating layer contains plasticizers and may also include inert excipients such as talc, titanium dioxide, colloidal silicon dioxide, hydroxypropyl methylcellulose and crospovidone Such plasticizers are for instance, but not limited to, triacetin, citric acid esters, phthalic acid esters, dibutyl sebacate, cetyl alcohol, polyethylene glycols, polysorbates or other plasticizers. The plasticizer is used in an amount of less than about 10% by weight of the enteric coating layer more preferably the plasticizer is about 8% by weight of the enteric coating polymer. Surprisingly, we have found that by using CEOLUS™ KG as one of the diluents the acid resistance of the multiple unit tablets is not compromised even if we reduce the plasticizer content to less than about 8%.

The core covered with enteric coating layer may further be covered with one or more over-coating layers. The materials for over-coating layers are pharmaceutically acceptable compounds such as but not limited to sugar, polyethylene glycol, polyvinylpyrrolidone, polyvinyl alcohol, polyvinyl acetate, hydroxypropyl cellulose, methylcellulose, ethylcellulose, hydroxypropylmethylcellulose and carboxymethylcellulose sodium, used alone or in mixtures. Additives such as plasticizers, colorants, pigments, fillers, anti-tacking and antistatic agents, such as magnesium stearate, titanium dioxide and talc may also be included into the over-coating layer. Said over-coating layer may further prevent potential agglomeration of enteric coating layered core, protect the enteric coating layer towards cracking during the compaction process and enhance the tableting process.

These coating layers comprises one or more excipients selected from the group comprising but not limited to coating agents, opacifiers, taste-masking agents, diluents, polishing agents, colouring agents, antitacking agents and the like.

Pharmaceutical composition of the invention can be coated by a wide variety of methods. Suitable methods include compression coating, coating in a fluidized bed or a pan and hot melt (extrusion) coating. Such methods are well known to those skilled in the art.

Another embodiment is a multiple unit tablet composition comprising:

(i) a enteric coated multiple unit cores comprising a pharmaceutically active ingredient, wherein plasticizer content of enteric coating is less than about 10% by weight of the enteric coating polymer,

(ii) atleast two diluents and optionally one or more pharmaceutically acceptable excipient(s), wherein one diluent is highly compactible microcrystalline cellulose, wherein the multiple units of the active ingredient are about 20-45% of the tablet weight.

It has been found that by using the multiple units to about 20-45% of the total tablet weight helps in avoiding the segregation and aids in proper flow of the powder from the hopper thereby maintaining the content uniformity in the multiple unit tablet composition.

According to yet another embodiment a multiple unit tablet composition comprising:

(i) enteric coated multiple unit cores comprising a pharmaceutically active ingredient, wherein plasticizer content of enteric coating is less than about 10% by weight of the enteric coating polymer,

(ii) atleast two diluents, one or more stabilizers and optionally one or more pharmaceutically acceptable excipient(s), wherein one diluent is highly compactible microcrystalline cellulose, wherein the multiple units of the active ingredient are about 20-45% of the tablet weight, and the multiple unit tablet composition has mechanical properties wherein the acid resistance is not compromised.

Stabilizer can be chosen among, but are not limited to, substances such as meglumine; L-cysteine hydrochloride; glycine hydrochloride; malic acid; sodium metabisulfate; citric acid, tartaric acid and L-cysteine dehydrochloride. sodium, potassium, calcium, magnesium and aluminum salts of phosphoric acid, carbonic acid, citric acid or other suitable weak inorganic or organic acids; aluminum hydroxide/sodium bicarbonate co precipitate; substances normally used in antacid preparations such as aluminum, calcium and magnesium hydroxides; magnesium oxide or composite substances, such as Al₂O₃ , 6MgO·CO₂·12H₂O, (Mg₆Al₂(OH)₁₆ CO₃ 4H₂ O), MgO·Al₂O₃, 2SiO₂·nH₂O or similar compounds; organic pH-buffering substances such as trihydroxymethylaminomethane, basic amino acids and their salts or other similar, pharmaceutically acceptable pH-buffering substances. The most preferred stabilizer is alkaline agent which helps in increasing the pH of the microenvironment and further helps in increasing the stability of the composition.

According to yet another embodiment a process for the preparation of multiple unit tablet composition comprising the steps of mixing enteric coated multiple unit cores of active ingredient having plasticizer content of less than about 10% by weight of the enteric coating polymer with atleast two diluents having highly compactible microcrystalline cellulose as one diluent and one or more other pharmaceutically acceptable excipients and compressed.

The pharmaceutical composition of the invention can be formed by various methods known in the art such as by dry granulation, wet granulation (aqueous, non-aqueous, hydroalcoholic), melt granulation, direct compression, dry granulation, double compression, extrusion spheronization, layering and the like.

The solvent(s) used in wet granulation in the present invention include all the solvents well known in the art or their mixtures thereof.

The most preferable process used for preparation of multiple unit tablet composition is non-aqueous and thus avoids the laborious process of drying the granules obtained in each step which further helps in avoiding the loss of active and thus improving the content uniformity.

The following examples are illustrative of the present invention, and the examples should not be considered as limiting the scope of this invention in any way, as these examples and other equivalents thereof will become apparent to those versed in the art, in the light of the present disclosure, and the accompanying claims

### EXAMPLES

### Preparation of Over Coated Pellets

### Example 1

| Sr. | | | |
|---|---|---|---|
| No. | Formula | mg/tab | stage |
| 1. | Sugar Spheres | 32 | Core |
| 2. | Esomeprazole Mg DH | 41.42 | |
| 3. | PVP | 10 | |
| 4. | Tween 80 | 0.15 | |
| 5. | Methanol | qs | |
| 6. | HPMC | 24 | Separating |
| 7. | PEG | 2.4 | Layer |
| 8. | Iso propyl alcohol (IPA) | qs | |
| 9. | Dichloromethane (DCM) | qs | |
| 10. | Eudragit | 67.05 | Enteric |
| 11. | Talc | 13.41 | coat |
| 12. | Tri ethyl citrate (TEC) | 6.03 | |
| 13. | Iso propyl alcohol | qs | |
| 14. | Dichloromethane | qs | |
| 15. | Hydroxy propyl methyl | 9.612 | Over |
| | cellulose(HPMC) | | coating |
| 16. | Talc | 1.44 | |
| 17. | Iso propyl alcohol | qs | |
| 18. | Dichloromethane | qs | |

### Example 2

| Sr. | | | |
|---|---|---|---|
| No. | Formula | mg/tab | stage |
| 1. | Sugar Spheres | 29.93 | Core |
| 2. | Esomeprazole Mg DH | 41.42 | |
| 3. | PVP | 8.28 | |
| 4. | Tween 80 | 0.2 | |
| 5. | Methanol | qs | |
| 6. | HPMC | 24 | Separating |
| 7. | PEG | 2 | Layer |
| 8. | Iso propyl alcohol | qs | |
| 9. | Dichloromethane | qs | |
| 10. | Eudragit | 67.05 | Enteric coat |
| 11. | Talc | 35.708 | |
| 12. | Tri ethyl citrate | 5.36 | |
| 13. | Iso propyl alcohol | qs | |
| 14. | Dichloromethane | qs | |
| 15. | Hydroxy propyl methyl cellulose | 9.612 | Over coating |
| 16. | Talc | 1.44 | |
| 17. | Iso propyl alcohol | qs | |
| 18. | Dichloromethane | qs | |

### Process of Preparation of Over Coated Pellets:

1. Dissolved PVP, Tween 80 and API in Methanol
2. Spray the solution of Step 2 on Sugar Spheres to get the drug-loaded pellets.
3. Dissolve PEG and HPMC in IPA and DCM mixture
4. Spray the solution of Step 3 on Step 2 drug loaded pellets to get barrier-coated pellets.
5. Dissolve Eudragit and TEC in IPA-DCM mixture and disperse Talc and Tween 80 in the same
6. Spray the solution of Step 5 on Step 4 barrier coated pellets to get the enteric-coated pellets.
7. Dissolve HPMC in IPA-DCM mixture and disperse talc in the same.
8. Spray the solution of Step 7 on Step 6 enteric coated pellets to get the over-coated pellets.

### Preparation of Multiple Unit Tablets Using Overcoated Pellets of Example 2

### Example 3

| S. No. | Ingredients | mg/tab |
|---|---|---|
| 1. | Over coated pellets | 191.8 |
| 2. | Silicified Microcrystalline cellulose | 64 |
| 3. | Microcrystalline cellulose 500 | 225.2 |
| 4. | Crospovidone | 22 |
| 5. | Magnesium Carbonate | 8 |
| 6. | Hydrogenated vegetable oil | 12 |
| 7 | Microcrystalline Cellulose | 7 |
| 8. | Microcrystalline Cellulose (Ceolus KG- | 50 |
| | 802) | |

### Example 4

| S. No. | Ingredients | mg/tab |
|---|---|---|
| 1. | Over coated pellets | 225 |
| 2. | Silicified Microcrystalline cellulose | 64 |
| 3. | Microcrystalline cellulose 500 | 192 |
| 4. | Crospovidone | 22 |
| 5. | Magnesium Carbonate | 8 |
| 6. | Hydrogenated vegetable oil | 12 |
| 7 | Microcrystalline Cellulose | 7 |
| 8. | Microcrystalline Cellulose (Ceolus KG- | 50 |
| | 802) | |

### Example 5

| S. No. | Ingredients | mg/tab |
|---|---|---|
| 1. | Over coated pellets | 261 |
| 2. | Silicified Microcrystalline cellulose | 64 |
| 3. | Microcrystalline cellulose 500 | 156 |
| 4. | Crospovidone | 22 |
| 5. | Magnesium Carbonate | 8 |
| 6. | Hydrogenated vegetable oil | 12 |
| 7 | Microcrystalline Cellulose | 7 |
| 8. | Microcrystalline Cellulose (Ceolus KG- | 50 |
| | 802) | |

### Example 6

| S. No. | Ingredients | mg/tab |
|---|---|---|
| 1. | Over coated pellets | 191.8 |
| 2. | Silicified Microcrystalline cellulose | 70 |
| 3. | Microcrystalline cellulose 500 | 224.2 |
| 4. | Crospovidone | 22 |
| 5. | Magnesium Carbonate | 8 |
| 6. | Hydrogenated vegetable oil | 12 |
| 7 | Microcrystalline Cellulose | 7 |
| 8. | Microcrystalline Cellulose(Ceolus KG- | 45 |
| | 802) | |

### Example 7

| S. No. | Ingredients | mg/tab |
|---|---|---|
| 1. | Over coated pellets | 225 |
| 2. | Silicified Microcrystalline cellulose | 100 |
| 3. | Microcrystalline cellulose 500 | 168 |
| 4. | Crospovidone | 15 |
| 5. | Magnesium Carbonate | 8 |
| 6. | Hydrogenated vegetable oil | 12 |
| 7 | Microcrystalline Cellulose | 7 |
| 8. | Microcrystalline Cellulose(Ceolus KG- | 45 |
| | 802) | |

### Example 8

| S. No. | Ingredients | mg/tab |
|---|---|---|
| 1. | Over coated pellets | 261 |
| 2. | Silicified Microcrystalline cellulose | 90 |
| 3. | Microcrystalline cellulose 500 | 156 |
| 4. | Crospovidone | 20 |
| 5. | Magnesium Carbonate | 8 |
| 6. | Hydrogenated vegetable oil | 12 |
| 7 | Microcrystalline Cellulose | 7 |
| 8. | Microcrystalline Cellulose (Ceolus KG- | 60 |
| | 802) | |

### Process for Preparation of Multiple Unit Tablets:

1. Mix the weighed quantity of over coated pellets, SMCC, Hydrogenated vegetable oil, MCC 500, Crospovidone, magnesium carbonate, Avicel PH 101 & MCC (Ceolus KG-802)in a cone blender for 20 min.
2. Compress the above blend into tablet and film coat the tablets with Opadry.

### Acid Resistance:

Dissolution of the multiple unit tablets was carried out in 900 ml of 0.1N HCl at 100 RPM using USP Type II (Paddle) apparatus for 2 hours. The results are as follows:

| | | Exam- | | Exam- | | Exam- |
|---|---|---|---|---|---|---|
| | Example 3 | ple 4 | Example 5 | ple 6 | Example 7 | ple 8 |
| Unit 1 | 0.9 | 4 | 3 | 5 | 3 | 4 |
| Unit 2 | 2 | 5 | 4 | 4 | 4 | 6 |
| Unit 3 | 3 | 3 | 5 | 7 | 5 | 3 |
| Unit 4 | 3 | 4 | 4 | 6 | 4 | 2 |
| Unit 5 | 4 | 2 | 6 | 6 | 6 | 1 |
| Unit 6 | 5 | 1 | 0.8 | 2 | 6 | 4 |
| Average | 2.98 | 3.16 | 3.8 | 5 | 4.66 | 3.33 |
| % drug | | | | | | |
| release | | | | | | |

1. A multiple unit tablet composition comprises:
(i) enteric coated multiple unit cores comprising a pharmaceutically active ingredient, wherein plasticizer content of enteric coating is less than about 10% by weight of the enteric coating polymer;
(ii) atleast two diluents and optionally one or more other pharmaceutically acceptable excipient, wherein one diluent is highly compactable microcrystalline cellulose.

2. The multiple unit tablet composition according to claim 1, wherein the pharmaceutically active ingredient is a benzimidazole derivative.

3. The multiple unit tablet composition according to claim 2, wherein the benzimidazole derivative is a proton pump inhibitor.

4. The multiple unit tablet composition according to claim 1, wherein said multiple units comprises separating layer(s) between the core and the enteric coating layer.

5. The multiple unit tablet composition according to claim 1, wherein one diluent is selected from confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, or combinations thereof.

6. The multiple unit tablet composition according to claim 1, wherein one or more pharmaceutically acceptable excipient selected from binders, diluents, lubricants, surfactants or glidants.

7. The multiple unit tablet composition according to claim 1, wherein the multiple units containing the active ingredient constitute about 20-45% of the total tablet weight.

8. The multiple unit tablet composition according to claim 1, wherein the enteric coated multiple unit cores release less than 10% of active ingredient in first 2 hours.

9. A process for the preparation of multiple unit tablet composition comprising steps of mixing enteric coated multiple unit cores of active ingredient having plasticizer content of less than about 10% by weight of the enteric coating polymer with, atleast two diluents having high compactible microcrystalline cellulose as one diluent and optionally one or more other pharmaceutically acceptable excipients and compressed.
